(19) United States
(12) Patent Application Publication
(10) Pub. No.: US 2025/0223657 A1
(43) Pub. Date: Jul. 10, 2025

(54) IDENTIFICATION OF A UNIQUE BACTERIAL STRAIN THAT CONFERS RISK OF RHEUMATOID ARTHRITIS AND RELATED MATERIALS AND METHODS

(71) Applicants: THE REGENTS OF THE UNIVERSITY OF COLORADO, a body corporate, Denver, CO (US); THE BOARD OF TRUSTEES OF THE LELAND STANFORD JUNIOR UNIVERSITY, Stanford, CA (US)

(72) Inventors: Kristine KUHN, Denver, CO (US); Meagan CHRISWELL, Aurora, CO (US); Michael V. HOLERS, Denver, CO (US); Brendan ALLEN, Aurora, CO (US); William H. ROBINSON, Stanford, CA (US)

(21) Appl. No.: 18/880,133

(22) PCT Filed: Jun. 30, 2023
(86) PCT No.: PCT/US2023/069489

§ 371 Date: Jan. 22, 2025

Related U.S. Application Data
(60) Provisional application No. 63/357,590, filed on Jun. 30, 2022.

(51) Int. Cl. C12Q 1/689

(52) U.S. Cl. CPC C12Q 1/689; C12Q 2600/156

(57) ABSTRACT

Methods for the early detection of subjects at risk of developing rheumatoid arthritis, and subjects having early rheumatoid arthritis thus allowing for early intervention.

### STATEMENT REGARDING FEDERALLY SPONSORED RESEARCH

This invention was made with government support under Grant Nos. U01AI101981, R01 AR075033, and P30 AR079369 awarded by the National Institutes of Health. The government has certain rights in the invention.

### SEQUENCE LISTING

The instant application contains a Sequence Listing which has been submitted electronically in XML format created on Jun. 30, 2023, named 2848-348-PCT.xml is 2,513,000 bytes in size, and is hereby incorporated by reference in its entirety.

### BACKGROUND

Rheumatoid arthritis (RA) is a progressive, systemic autoimmune disease characterized by chronic inflammation of multiple joints with associated symptoms such as fatigue. The inflammation causes joint pain, stiffness, and swelling resulting in loss of joint function due to destruction of bone and cartilage. RA often leads to progressive disability. Patients with RA also have an increased likelihood of developing other systemic complications such as osteoporosis, anemia, cardiovascular disease, and disorders affecting the lungs and skin. The disease shortens the average life expectancy of affected patients by three to seven years. About 0.5-1% of the US adult population is affected by RA, and given the average age of onset is approximately 50, the number of affected individuals is expected to rise. Despite declining rates of disability attributed to RA, about 30% of patients do not achieve a minimally acceptable symptom state.

Early treatment of RA is associated with decreased joint damage and disability. In addition, studies performed in individuals considered to have pre-RA, defined as clinical risk for RA but not yet exhibiting classifiable disease, have demonstrated only marginally effective prevention of clinical RA onset. In addition, identification of individuals at risk for developing RA is a challenging and inexact process. Currently, prediction tools for the development of RA are based on circulating antibodies and genetic factors. These tools can classify only 30-40% of individuals with RA as high risk of developing RA in the next 2-3 years.

New methods are needed for identifying individuals at risk for developing RA or in an early stage of RA, and new therapeutic strategies are needed in the pre-RA period to prevent the onset of clinical disease.

### SUMMARY

The mucosal origins hypothesis of rheumatoid arthritis (RA) development proposes a central role for mucosal immune responses in the initiation and/or perpetuation of the systemic autoimmunity that occurs with disease and begins well before arthritis develops. As described in WO 2022/099317, using multiple independent methods, a novel species and strain of Subdoligranulum—Subdoligranulum didolesgii strain D8 (also referred to as isolate 7 throughout this disclosure)—was identified as being sufficient to cause rheumatoid arthritis (RA) or to worsen its disease course and effects on involved tissues.

As shown herein, S. didolesgii strain D8 is present in the feces of individuals at risk of RA and in individuals with early RA. Accordingly, the presence and/or abundance of Subdoligranulum didolesgii strain D8 in a human subject, such as in a fecal sample or other biological sample disclosed herein, can be used as a diagnostic indicator for RA, and as a predictive indicator for risk of RA onset. The present disclosure is directed to methods for diagnosing a subject with RA and for identifying a subject at risk of developing RA. The methods comprise detecting the presence or absence of Subdoligranulum didolesgii strain D8, in a biological sample of a subject. The methods advantageously permit early therapeutic action that may contribute to the slowing or prevention of tissue damage caused by RA. The skilled artisan will recognize other benefits of the disclosed methods and materials.

### BRIEF DESCRIPTION OF THE DRAWINGS

FIGS. 1A, 1B, and 1C depict data demonstrating that Subdoligranulum isolate 7 is detectable in the feces of individuals in the at-risk period and early stages of RA. FIG. 1A shows a standard curve created by spiking a human fecal sample with known quantities (colony forming units (CFU)/ml) of Subdoligranulum isolate 7 (designated Subdoligranulum didolesgii strain D8) The curve ranges from 1×10⁹ CFU of isolate 7 per 100 mg of feces to 0 CFU of isolate 7, with a limit of detection set at 1×10⁵ CFU per 100 mg of feces. A line of best fit was set for regression analysis (R²=0.9982). FIG. 1B shows qPCR analysis for the presence of isolate 7 in feces from healthy controls (n=12), individuals at-risk for RA (n=12), individuals with early RA (n=12) or specific pathogen-free (SPF) mice (n=12). The number and percentage of samples above the limit of detection for the assay is displayed by group (***, P<0.001, Chi-square test). FIG. 1C shows the results of a regression analysis performed utilizing the line of best fit for the assay to determine the estimated CFUs of isolate 7 in the positive samples. The mean CFUs per 100 mg feces is displayed ±SEM for all positive samples (n=4), for positive samples from individuals at-risk for RA (n=2) and for positive samples for individuals with early RA (n=2).

FIG. 2 is a table of characteristics of human fecal samples for Subdoligranulum isolate 7-specific qPCR data shown in FIG. 1.

FIG. 3 depicts estimated percentage of isolate 7 out of total bacterial load in human fecal samples from those individuals listed in FIG. 2. The estimated percentage of total bacteria per 100 mg feces that is isolate 7 was estimated for each positive sample, based on a previously published estimate of total CFUs of bacteria per fecal weight (Stephen, 1980, J Med Microbiol 13:45-56). The mean percentage of bacteria that are isolate 7 is displayed ±SEM for all positive samples (n=4), for positive samples from individuals at risk for RA (n=2), and for positive samples for individuals with early RA (n=2).

FIG. 4 provides a summary of the demographics of three groups of human subjects: healthy controls, individuals at-risk for RA, and individuals with early RA for the data shown in FIGS. 5 and 6. Kruskal-Wallis and Mann-Whitney testes were used for 3- and 2-way comparisons of continuous data, respectively. Categorical data comparisons were performed using Chi-square or Fisher's exact test. Correlations were performed using Spearman's rank correlation coefficient.

FIG. 5 shows the fecal concentration of S. dido 7 (in ng/μL) significantly differed across study three groups by Kruskal-Wallis test (p-0.02). After adjustment for multiple comparisons, the At-Risk group had a significantly higher fecal concentration of S. dido 7 compared to the control group by Mann-Whitney test (p=0.024).

FIG. 6 shows Spearman correlations between three biomarkers and subject age and BMI with fecal concentration of S. dido 7.

### DETAILED DESCRIPTION

In the following sections, various compositions and methods are described to detail various embodiments. Practicing the various embodiments does not require the employment of all the specific details outlined herein, but rather concentrations, times, and other specific details may be modified. In some cases, well known methods or components have not been included in the description.

A “subject” means any individual having rheumatoid arthritis (RA), having symptoms of RA, or at risk for developing RA. A subject may be human or non-human, and may include, for example, animals or species used as “model systems” for research purposes, such as a mouse model. The subject may be a human patient having RA or at risk of developing RA.

As used herein, “at-risk of developing RA” is defined as an individual that is a first-degree relative (FDR) of a patient with RA or an individual with preclinical RA. “Preclinical RA” refers to the presence of positive levels of circulating RA-specific autoantibodies, i.e., serum anti-cyclic citrullinated peptide positive (CCP+) and/or RF positive (RF+) and/or other antibodies to modified protein antigens (AMPA) and/or biomarkers that define the preclinical state of seronegative RA. Circulating RA-specific autoantibodies refers to serum anti-CCP3/anti-CCP3.1 positive and/or ≥2 RF isotypes.

As used herein, “having RA” is defined as an individual diagnosed using American College of Rheumatology (ACR) 1987 and/or ACR European League Against Rheumatism (EULAR) 2010 classification criteria; such criteria include exhibiting clinical symptoms and clinical signs and biomarkers of rheumatoid arthritis. As used herein, “early RA” refers to an individual within one year of RA diagnosis.

The present disclosure provides methods for identifying subjects at risk of developing RA, having pre-RA, having preclinical RA, having clinically suspect arthralgia (CSA), having early RA, or having RA. Such methods generally include detecting the presence or absence of the disclosed Subdoligranulum sp. nov. in the subject, with the presence of the Subdoligranulum didolesgii strain D8 being linked to a risk of developing RA or indicative of pre-RA, early RA, or RA in the subject. Also provided are methods for preventing development and/or progression of RA in a subject, or for treating RA in a subject. Such methods generally involve administering to a subject in need of treatment one or more therapeutic agents.

As used herein, “treat” in reference to a condition means: (1) to ameliorate or prevent the condition or one or more of the biological manifestations of the condition, (2) to interfere with (a) one or more points in the biological cascade that leads to or is responsible for the condition or (b) one or more of the biological manifestations of the condition, (3) to alleviate one or more of the symptoms or effects associated with the condition, and/or (4) to slow the progression of the condition or one or more of the biological manifestations of the condition. The terms “prevent,” “preventing,” and the like are to be understood to refer to a method of blocking the onset of disease and/or its attendant symptoms. “Prevent” also encompasses delaying or otherwise impeding the onset of a disease and/or its attendant symptoms.

As used herein, “therapeutically effective amount” in reference to an agent means an amount of the agent sufficient to treat the subject's condition but low enough to avoid serious side effects at a reasonable benefit/risk ratio within the scope of sound medical judgment. The safe and effective amount of an agent will vary with the particular agent chosen (e.g. consider the potency, efficacy, and half-life of the compound); the route of administration chosen; the condition being treated; the severity of the condition being treated; the age, size, weight, and physical condition of the patient being treated; the medical history of the patient to be treated; the duration of the treatment; the nature of concurrent therapy; the desired therapeutic effect; and like factors, but can nevertheless be determined by the skilled artisan.

The inventors previously identified a specific bacterial strain in the family Ruminococcus genus Subdoligranulum that is targeted by autoantibodies of individuals at risk for RA and those with classified RA. The Subdoligranulum sp. nov. and, in particular, Subdoligranulum didolesgii strain D8, is demonstrated herein as a biomarker of risk of RA or early RA. The strain, designated Subdoligranulum didolesgii strain D8, is maintained in the lab of inventor Kuhn and was deposited with the ATCC Patent Depository under the Budapest Treaty (10801 University Blvd, Manassas, VA 20110), on 9 Nov. 2021, with the ATCC Patent Deposit Designation No. PTA-126891. A genome contig of S. didolesgii strain D8 is provided in SEQ ID NO: 1. Two specific regions of the genome contig are provided: SEQ ID NO: 2 (referred to herein as “Region A”) and SEQ ID NO: 3 (referred to herein as “Region B”), as follows:

Region A (233 bp):
(SEQ ID NO: 2)
atttaaaataaataaatataatggaatagatgtattaaaaagaaatataa
ctaccgaaaacacacacataaacacttaatagtattttataactatcgag
tgggaatgatatcacactcgcctgaaagccagtaaccgtgcgggttacac
cgttctggatatatcttgtggcaacgttttggcaacacctgcaacataaa
ttgtattgttccataaatatagagctaactgaa
Region B (1443 bp):
(SEQ ID NO: 3)
ttgaatagagttatcttaatattatttcttttagatttgtttgtatcaac
atgctcttttctaagttgaattatattattactgtctgatgtaactagaa
cagttataacagctatcgaaaatgataacaatatagcaattgaatttatt
tgaatgtctataaatgattgaaacaacttaggtaagtatattttatctct
agacagaattacaatcaaagtaagcactaaagcaattaaccctgagatta
acatacctacccatattttgtccatatatttgtaataatcgtacaccggt
aatagtatagttttttttacaaatgctttaaacctcaacactttcacctc
attccaattttaattgttctaataattgctgagccttttgaaagaagtca
gcgctgtctacctcgttagtatcagatgtagtttcaacaataattttttc
ttttttcttaagttgttccgtatctaaaattgtatctccacctgatttta
cacataatctctttacttcattctcgttattatcaataagattataatat
ctctttatagcatctttagatatatatggactttttcgtccctttcgttt
aaaaataatatcaacatattcagacatatcatctctatcagcaaattgta
aatacttatcatcaagagaatttctttcaacagttaactttaaaagagta
aacttttttgcaatattaagtttagctaaaaacccttcatccggaataaa
atcactcgaaaaactatacctatactcatgattactaatatctgcaaaat
ttttcattttatgatataaataatttacaatttgacttattcctataccg
ttataatttttttcaaacatacataaaaatgtattacaccccttattatg
tctaatacataaatgagtgcgttcttcatcaccatcaaattcacctttga
gtattcctaaattatccatagttgaattatttcgaacatttcttctataa
ttatacttaagtgacttaaagattatattataatttctgttatccaatgc
ctcaaaagagtccatccatagagtcttatttttatttggatattctattc
ttctttcgaccaatgtttggttagacaaattattcattgcatcaacaaaa
aaaggtataaaatcctcaatagtaccttctggtaaatcaggcgaagtagc
cgtaatagttaaatttaagtaataaaaaccgacagagacatttttattca
tacatatatcctccaaattaacaaaatattatttatctagataatacaat
attttaaaataaatgtcaatatatgtaaaatatatcttaaactctataaa
atatttattatagtacattatataaccaatcttgtgtcagtgcgtgccaa
aaaaataaaggcgtaacaaatttatttgttctgcccttactat

With regard to S. didolesgii strain D8, the term “polynucleotide” as used in accordance with the present disclosure relates to a polynucleotide comprising a nucleic acid sequence of SEQ ID NO: 1 or a variant of SEQ ID NO: 1, a nucleic acid sequence of SEQ ID NO: 2 or a variant of SEQ ID NO: 2, or a nucleic acid sequence of SEQ ID NO: 3 or a variant of SEQ ID NO: 3. The polynucleotide variants, preferably, comprise a nucleic acid sequence characterized in that the sequence can be derived from SEQ ID NO: 1, 2, or 3 by at least one nucleotide substitution, addition and/or deletion. Variants also encompass polynucleotides comprising a nucleic acid sequence which is capable of hybridizing to the aforementioned specific nucleic acid sequences, preferably, under stringent hybridization conditions. These stringent conditions are known to the skilled worker and can be found in Current Protocols in Molecular Biology, John Wiley & Sons, N. Y. (1989), 6.3.1-6.3.6. A preferred example for stringent hybridization conditions are hybridization conditions in 6× sodium chloride/sodium citrate (=SSC) at approximately 45° C., followed by one or more wash steps in 0.2×SSC, 0.1% SDS at 50 to 65° C. The skilled worker knows that hybridization conditions differ depending on the type of nucleic acid and, for example when organic solvents are present, the temperature and concentration of the buffer.

Percent identity values are generally calculated over the entire nucleic acid sequence region. A series of programs based on a variety of algorithms is available to the skilled worker for comparing different sequences.

A mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site having the universal resource locator “www.ncbi.nlm.nih.gov/BLAST/”. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences having sequence identity to a nucleic acid described herein.

Percent identity between two nucleotide sequences may be determined using the needle program in the EMBOSS software package (EMBOSS: The European Molecular Biology Open Software Suite, Rice, P., Longden, I., and Bleasby, A., Trends in Genetics 16 (6), 276-277, 2000), using the EDNAFULL scoring matrix and a gap opening penalty of 16, 14, 12, 10, 8, 6, or 4 and a gap extension penalty of 0.5, 1, 2, 3, 4, 5, or 6.

To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (id.) and relationships between molecules which share a common pattern. When utilizing BLAST, Gapped BLAST, PSI-Blast, and PHI-Blast programs, the default parameters of the respective programs (e.g., XBLAST and NBLAST) can be used. See ncbi.nlm.nih.gov.

The percent identity between two sequences can be determined using techniques similar to those described above, with or without allowing gaps. In calculating percent identity, typically exact matches are counted.

Variants encompassed by the present disclosure include polynucleotides comprising nucleic acid sequences which are at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3. at least 99.4%, at least 99.5%, at least 99.6%, at least 99.7%, at least 99.75%, at least 99.8%, at least 99.85%, or at least 99.9% identical to SEQ ID NO: 1, 2 or 3. Variants encompassed by the present disclosure have up to about 25,000 nucleotide differences (e.g., substitutions, additions and/or deletions) compared to SEQ ID NO: 1. Variants encompassed by the present disclosure have up to 20,000 nucleotide differences compared to SEQ ID NO: 1, up to 10,000, up to 7500, up to 7525, up to 7000, up to 6500, up to 6000, up to 5500, up to 5000, up to 4500, up to 4000, up to 3500, up to 3000, up to 2500, up to 2000, up to 1500, or up to 1000 nucleotide differences compared to SEQ ID NO: 1. Variants encompassed by the present disclosure have up to about 50 nucleotide differences (e.g., substitutions, additions and/or deletions) compared to SEQ ID NO: 2. Variants encompassed by the present disclosure have up to 35 nucleotide differences compared to SEQ ID NO: 2, up to 25, up to 20, up to 19, up to 16, up to 14, up to 12, up to 10, up to 7, up to 5, up to 3, up to 2, up to 3000, or up to 1 nucleotide differences compared to SEQ ID NO: 2. Variants encompassed by the present disclosure have up to about 30 nucleotide differences (e.g., substitutions, additions and/or deletions) compared to SEQ ID NO: 3. Variants encompassed by the present disclosure have up to 225 nucleotide differences compared to SEQ ID NO: 3, up to 200, up to 150, up to 145, up to 130, up to 115, up to 100, up to 85, up to 70, up to 60, up to 45, up to 30, up to 15, up to 10, up to 8, up to 5, or up to 2 nucleotide differences compared to SEQ ID NO: 3.

Variants encompassed include Subdoligranulum didolesgii strains comprising nucleic acid sequences which are at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3. at least 99.4%, at least 99.5%, at least 99.6%, at least 99.7%, at least 99.75%, at least 99.8%, at least 99.85%, or at least 99.9% identical to SEQ ID NO: 1, 2 or 3.

Variants encompassed include Subdoligranulum didolesgii strains comprising nucleic acid sequences which have up to about 25,000 nucleotide differences compared to SEQ ID NO: 1. Variants encompassed by the present disclosure include Subdoligranulum didolesgii strains having up to 20,000 nucleotide differences compared to SEQ ID NO: 1, up to 10,000, up to 7500, up to 7525, up to 7000, up to 6500, up to 6000, up to 5500, up to 5000, up to 4500, up to 4000, up to 3500, up to 3000, up to 2500, up to 2000, up to 1500, or up to 1000 nucleotide differences compared to SEQ ID NO: 1. Variants encompassed by the present disclosure include Subdoligranulum didolesgii strains having up to 20,000 nucleotide substitutions compared to SEQ ID NO: 1, up to 10,000, up to 7500, up to 7525, up to 7000, up to 6500, up to 6000, up to 5500, up to 5000, up to 4500, up to 4000, up to 3500, up to 3000, up to 2500, up to 2000, up to 1500, or up to 1000 nucleotide substitutions compared to SEQ ID NO: 1.

Variants encompassed by the present disclosure include Subdoligranulum didolesgii strains having up to about 50 nucleotide differences compared to SEQ ID NO: 2. Variants encompassed by the present disclosure include Subdoligranulum didolesgii strains having up to 35 nucleotide differences compared to SEQ ID NO: 2, up to 25, up to 20, up to 19, up to 16, up to 14, up to 12, up to 10, up to 7, up to 5, up to 3, up to 2, up to 3000, or up to 1 nucleotide differences compared to SEQ ID NO: 2. Variants encompassed by the present disclosure include Subdoligranulum didolesgii strains having up to about 50 nucleotide substitutions compared to SEQ ID NO: 2. Variants encompassed by the present disclosure include Subdoligranulum didolesgii strains having up to 35 nucleotide substitutions compared to SEQ ID NO: 2, up to 25, up to 20, up to 19, up to 16, up to 14, up to 12, up to 10, up to 7, up to 5, up to 3, up to 2, up to 3000, or up to 1 nucleotide substitutions compared to SEQ ID NO: 2.

Variants encompassed by the present disclosure include Subdoligranulum didolesgii strains having up to about 30 nucleotide differences compared to SEQ ID NO: 3. Variants encompassed by the present disclosure include Subdoligranulum didolesgii strains having up to 225 nucleotide differences compared to SEQ ID NO: 3, up to 200, up to 150, up to 145, up to 130, up to 115, up to 100, up to 85, up to 70, up to 60, up to 45, up to 30, up to 15, up to 10, up to 8, up to 5, or up to 2 nucleotide differences compared to SEQ ID NO: 3. Variants encompassed by the present disclosure include Subdoligranulum didolesgii strains having up to about 30 nucleotide substitutions compared to SEQ ID NO: 3. Variants encompassed by the present disclosure include Subdoligranulum didolesgii strains having up to 225 nucleotide substitutions compared to SEQ ID NO: 3, up to 200, up to 150, up to 145, up to 130, up to 115, up to 100, up to 85, up to 70, up to 60, up to 45, up to 30, up to 15, up to 10, up to 8, up to 5, or up to 2 nucleotide substitutions compared to SEQ ID NO: 3. A variant strain may comprise at least one target antigen described herein. Alternatively or additionally, a variant strain may comprise at least one target antigen described herein. Alternatively or additionally, a variant strain may comprise at least one property selected from: causes local intestinal isolated lymphoid follicles (ILF) formation, stimulates T cell activation, induces development of RA-related autoantibodies, and induces joint swelling.

In methods of this disclosure, the presence of S. didolesgii strain D8, or a variant thereof, in a subject, identifies the subject as being at risk of developing rheumatoid arthritis, such as having preclinical RA, or as having RA, such as early RA.

Methods of Detecting S. didolesgii Strain D8

Provided herein are methods for detecting the presence of S. didolesgii strain D8 in a biological sample obtained from a subject. The inventors have created a method of detecting S. didolesgii Strain D8 with high specificity/discrimination. The method distinguishes between D8 and strain H3; strain H3 is another S. didolesgii Strain that is commonly found in fecal samples of individuals who have RA but is not suitable as a diagnostic biomarker. In addition, the method has unexpectedly high sensitivity in detecting S. didolesgii Strain D8 at an abundance of 0.0002% in feces. High specificity/discrimination is important to the use of strain D8 as a biomarker for risk assessment and diagnostic use.

Accordingly, a method for diagnosing rheumatoid arthritis (RA) in a subject is provided. The method comprises a) obtaining a biological sample from the subject; b) detecting presence or absence of Subdoligranulum didolesgii strain D8 in the biological sample, wherein the bacteria comprises SEQ ID NO: 2 (Region A) and/or SEQ ID NO: 3 (Region B), and c) diagnosing the subject with RA when Subdoligranulum didolesgii strain D8 bacteria is detected in the biological sample. Also provided is a method for diagnosing risk of rheumatoid arthritis (RA) in a subject. The method comprises a) obtaining a biological sample from the subject; b) detecting presence or absence of Subdoligranulum didolesgii strain D8 in the biological sample, wherein the bacteria comprises SEQ ID NO: 2 (Region A) and/or SEQ ID NO: 3 (Region B), and c) diagnosing the subject with RA when Subdoligranulum didolesgii strain D8 bacteria is detected in the biological sample. Detecting the presence of Subdoligranulum didolesgii strain D8 can be assessed relative to a control sample from a subject not diagnosed with RA and control sample from a subject not at risk of RA. Detection can also be a relative abundance of S. didolesgii Strain D8 above a threshold. Abundance may be assessed by assessing CFUs of S. didolesgii Strain D8 per weight or amount of biological sample. The threshold may be 2+SD (standard deviation) above the mean value for controls.

The presence of S. didolesgii strain D8 can be detected utilizing a genomic approach, where bacterial genomic DNA isolated from a biological sample is analyzed to determine its genetic identity. The genomic approach can involve, for example, PCR analysis or whole genome sequencing (WGS). Where WGS sequencing is utilized, methods can include a PCR step or a multiple displacement amplification step.

The presence of S. didolesgii strain D8 can be detected by PCR analysis, such as quantitative PCR (qPCR). As disclosed herein, thirty (30) different primer pairs were designed (10 sets for Region A and 20 sets for Region B) and tested for detection of S. didolesgii strain D8 in a biological sample (feces). Only three primer pairs had detection above the control. These three primer pairs are: primer pair 8, primer pair 7, and primer pair 16. Each of these three primer pairs are shown in the table below and for each primer pair, the forward primer is the plus template strand and the reverse primer is the minus template strand. Accordingly, the PCR analysis may include the use of primer pair 8, or the use of primer pair 7, or the use of primer pair 16. The PCR analysis may include the use of primer pair 8 and one of primer pair 7 and primer pair 16. The PCR analysis may include the use of primer pair 8, primer pair 7, and primer pair 16.

| Sequence | SEQ ID | | | |
|---|---|---|---|---|
| (5′->3′) | NO: | Length | Start | Stop |

PRIMER PAIR 8 (Region A)

| | | | | | |
|---|---|---|---|---|---|
| Forward | ACCGAAAACACAC | 4 | 24 | 53 | 76 |
| primer | ACATAAACACT | | | | |
| Reverse | GGTTACTGGCTTT | 5 | 20 | 137 | 118 |
| primer | CAGGCGA | | | | |

| | |
|---|---|
| Product | 85 |
| length | |

PRIMER PAIR 7 (Region A)

| | | | | | |
|---|---|---|---|---|---|
| Forward | GGGAATGATATCA | 6 | 22 | 102 | 123 |
| primer | CACTCGCCT | | | | |
| Reverse | TTGCCAAAACGTT | 7 | 21 | 185 | 165 |
| primer | GCCACAAG | | | | |

| | |
|---|---|
| Product | 84 |
| length | |

PRIMER PAIR 16 (Region B)

| | | | | | |
|---|---|---|---|---|---|
| Forward | GTCAGCGCTGTCT | 8 | 20 | 397 | 416 |
| primer | ACCTCGT | | | | |
| Reverse | ACGGCTACTTCGC | 9 | 19 | 1203 | 1185 |
| primer | CTGATT | | | | |

| | |
|---|---|
| Product | 807 |
| length | |

Direct sequence analysis can be used to detect a bacterial nucleic acid (e.g., a genome) of interest. A sample including DNA or RNA can be used, and PCR or other appropriate methods can be used to amplify all or a fragment of the nucleic acid, and/or its flanking sequences, if desired. The bacterial nucleic acid, or a fragment thereof, is determined, using standard methods.

Presence of S. didolesgii strain D8 can be detected by identifying the presence in the biological sample of S. didolesgii strain D8 genomic DNA. A contig representing the genomic sequence of S. didolesgii strain D8 is provided in SEQ ID NO: 1. An alignment of experimental sequences to SEQ ID NO: 1 can reveal the presence of S. didolesgii strain D8 in the sample. Experimental sequences may be aligned to SEQ ID NO: 2 or 3. S. didolesgii strain D8 is maintained in the lab of inventor Kuhn and was deposited with the ATCC Patent Depository under the Budapest Treaty (10801 University Blvd, Manassas, VA 20110), on 9 Nov. 2021. Those of skill in the art will be able to compare the deposited S. didolesgii strain D8 to bacteria—and their genomes—identified in biological samples.

Arrays of oligonucleotide probes complementary to target microbial nucleic acid sequences can be used to detect and identify microbial nucleic acids. The oligonucleotide probes may be complementary to sequences of Region A (SEQ ID NO: 2) and/or sequences of Region B (SEQ ID NO: 3).

Biological samples can include, for example, fecal samples, serum, plasma, peripheral blood cells, bone marrow, tissues including synovium or other joint-related tissues such as synovial fluid, sputum, rectal swabs, intestinal biopsies, along with samples from other mucosal or cutaneous sites. The biological sample may be a fecal sample.

Hybridization methods, such as Southern analysis, Northern analysis, or in situ hybridizations, can be used (see Current Protocols in Molecular Biology, Ausubel, F. et al., eds., John Wiley & Sons, including all supplements). For example, the presence of nucleic acid from a particular type of bacteria (e.g., S. didolesgii strain D8) can be determined by hybridization of nucleic acid to a nucleic acid probe. A “nucleic acid probe,” as used herein, can be a DNA probe or an RNA probe. The nucleic acid probe can be, for example, a full-length nucleic acid molecule, or a portion thereof, such as an oligonucleotide of at least 15, 30, 50, 100, 250 or 500 nucleotides in length and sufficient to specifically hybridize under stringent conditions to appropriate target RNA or DNA. Appropriate target sequences can include Region A (SEQ ID NO: 2), or fragments thereof, and/or Region B (SEQ ID NO: 3), or fragments thereof. The hybridization sample is maintained under conditions which are sufficient to allow specific hybridization of the nucleic acid probe to RNA or DNA. Specific hybridization can be performed under high stringency conditions or moderate stringency conditions, as appropriate. The hybridization conditions for specific hybridization are high stringency. More than one nucleic acid probe can also be used concurrently in this method. Specific hybridization of any one of the nucleic acid probes is indicative of the presence of the S. didolesgii bacterial strain of interest, as described herein.

Also provided are methods for preparing a sample fraction for detection of S. didolesgii strain D8 in a subject. Such methods generally include contacting a sample (e.g., fecal sample, fecal swab, or intestinal biopsy) with one or more monoclonal antibodies or one or more monoclonal antibody fragments having a variable region as described in WO 2022/099317. After contacting the sample with the one or more monoclonal antibodies or one or more monoclonal antibody fragments, the resulting fraction will include an increased relative abundance of bacteria from Lachnospiraceae, Ruminococcaceae, or both Lachnospiraceae and Ruminococcaceae in the sample fraction. Such methods can be set up similarly to a traditional immunoprecipitation assay, which will provide for the “pull down” of the target bacteria. After achieving the enriched sample fraction, the bacteria can be released from one or more monoclonal antibodies or one or more antibody fragments.

### Methods of Diagnosis

This disclosure provides methods of diagnosing a subject as having, or as being at risk for developing, rheumatoid arthritis. A subject can be diagnosed as having, or as being at risk for developing, rheumatoid arthritis by detecting the presence of S. didolesgii strain D8 in a subject. Presence of S. didolesgii strain D8 can be detected in accordance with the methods described herein.

The methods for diagnosing a subject as having, or as being at risk for developing, rheumatoid arthritis may further include effectuating a treatment regimen on a subject diagnosed as having, or at risk of having, RA. The method can include diagnosing a subject with RA or at risk of developing RA based upon the detection of S. didolesgii strain D8 in a subject, and administering a treatment regimen described herein to the subject to treat or otherwise prevent RA in the subject.

### Treatment Methods

Methods for treating rheumatoid arthritis in a subject, or ameliorating at least one symptom of rheumatoid arthritis in a subject, as known in the art. Methods of treatment can be executed by administering to a subject an effective amount of an agent known for treating RA.

These agents include but are not limited to non-steroidal anti-inflammatory drugs, corticosteroids, disease-modifying antirheumatic drugs, and biologic response modifiers.

These agents include but are not limited to non-steroidal anti-inflammatory drugs, corticosteroids, disease-modifying antirheumatic drugs, and biologic response modifiers.

Examples of suitable non-steroidal anti-inflammatory drugs include: ibuprofen; naproxen sodium; celecoxib; diclofenac; fenoprofen; flurbiprofen; indomethacin; ketorolac; mefenamic acid; meloxicam; oxaprozin; piroxicam; and sulindac.

Examples of suitable corticosteroids include: prednisone; bethamethasone; and prednisolone; triamcinolone; methylprednisolone; and dexamethasone.

Examples of suitable disease-modifying antirheumatic drugs include: methotrexate; leflunomide; hydroxychloroquine; and sulfasalazine.

Examples of suitable biologic response modifiers include: abatacept and biosimilars thereof; adalimumab and biosimilars thereof; anakinra and biosimilars thereof; baricitinib; certolizumab and biosimilars thereof; etanercept and biosimilars thereof; golimumab and biosimilars thereof; infliximab and biosimilars thereof; rituximab and biosimilars thereof; sarilumab and biosimilars thereof; tocilizumab and biosimilars thereof; and tofacitinib.

The therapeutic agents can be administered prophylactically or therapeutically to subjects at risk of (or susceptible to) developing RA, those presenting with preclinical RA, or those subjects diagnosed with RA. Prophylactic administration occurs prior to the manifestation of overt clinical symptoms of RA, such that RA is prevented or alternatively delayed in its progression slow the disease. Where a cure or prevention of the RA in a subject diagnosed to be at risk of developing RA, the goal of prophylactic treatment is to control symptoms and help the subject maintain a normal quality of life. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

Administration of the agents can be achieved in several ways using methods known in the art, including systemic administration (e.g., enteral or parenteral administration), intradermal delivery, intramuscular delivery, subcutaneous delivery, inhalation, and intranasal delivery.

The agents can be administered to a subject alone, or in conjunction with one or more agents, such as those traditionally utilized in the treatment of RA.

The agents may be administered to a subject as frequently as several times daily, or they may be administered less frequently, such as once a day, once a week, once every two weeks, once a month, or even less frequently, such as once every several months or even once a year or less. The frequency of the dose will be readily determinable by the skilled artisan and will depend upon any number of factors, such as, but not limited to, the type and severity of the disease being treated.

### Kits

Also provided are kits useful in carrying out the methods of this disclosure. Such kits comprise components useful in any of the methods described herein, including for example, primer pairs for qPCR, hybridization probes or primers (e.g., labeled probes or primers), reagents for detection of labeled molecules, means for amplification of nucleic acids, means for analyzing a nucleic acid sequence, antibodies, and instructional materials. For example, a kit may comprise components useful for analysis of a bacterial nucleic acid of interest present in a biological sample obtained from a subject. The kit may comprise components for detecting one or more of the bacterial nucleic acids of interest present in a biological sample derived from a subject. The kit may comprise primer pair 8 and optionally primer pair 7 and/or primer pair 16, as disclosed herein, for detecting bacterial nucleic acid of Subdoligranulum didolesgii strain D8 present in a biological sample derived from a subject.

### EXAMPLES

The materials, methods, and embodiments described herein are further defined in the following Examples. Certain embodiments are defined in the Examples herein. These Examples, while demonstrating certain embodiments, are given by way of illustration only. From the disclosure herein and these Examples, one skilled in the art can ascertain the essential characteristics of this invention, and without departing from the spirit and scope thereof, can make various changes and modifications of the invention to adapt it to various usages and conditions.

As described in the inventors' copending application (PCT/US2021/072293, filed 8 Nov. 2021; WO 2022/099317, published 12 May 2022), to investigate immune responses to Lachnospiraceae and Ruminococcaceae in RA, primary bacterial isolates from an individual with >40% abundance of these taxa in their feces were established. A total of 50 isolates were established, of which seven were identified as Lachnospiraceae/Ruminococcaceae by qPCR (isolates 1-7), and five were confirmed as pure isolates by 16S rRNA sequencing (isolates 1, 3-5, and 7). These five isolates underwent whole genome sequencing. The five isolates were narrowed to two candidates (isolates 1 and 7) for further study due to their differing abilities to induce joint swelling in monocolonized mice. Isolate 1 was designated Subdoligranulum didolesgii strain H3 and isolate 7 was designated Subdoligranulum didolesgii strain D8. Of these two candidates, Subdoligranulum didolesgii strain D8 (isolate 7), was identified as sufficient to cause rheumatoid arthritis (RA) or to worsen its disease course and effects on involved tissues.

### Example 1: Subdoligranulum Isolate 7 Specific qPCR

To determine the biological relevance of Subdoligranulum isolate 7 (S. didolesgii strain D8) in humans at-risk for early RA (<1 year from diagnosis), regions of the genome unique to isolate 7 and not present in isolate 1 were used to design a specific qPCR assay testing for the presence of isolate 7.

Two regions of genomic DNA present in Subdoligranulum isolate 7, but absent in isolate 1, were identified: Region A (SEQ ID NO: 2), supra and Region B (SEQ ID NO: 3), supra. From these regions, the NCBI primer design tool was utilized to generate 30 sets of potential isolate-7-specific primers; 10 primer sets were generated for Region A, as follows:

| | | | | |
|---|---|---|---|---|
| | Seq ID | | | |
| Sequence (5′->3′) | No. | Length | Start | Stop |

Primer pair 1

| | | | | | |
|---|---|---|---|---|---|
| Forward primer | TCGCCTGAAAGCCAGTAACC | 10 | 20 | 118 | 137 |
| Reverse primer | AGGTGTTGCCAAAACGTTGC | 11 | 20 | 190 | 171 |

| | |
|---|---|
| Product length | 73 |

Primer pair 2

| | | | | | |
|---|---|---|---|---|---|
| Forward primer | ACTCGCCTGAAAGCCAGTAA | 12 | 20 | 116 | 135 |
| Reverse primer | GTTGCCAAAACGTTGCCACA | 13 | 20 | 186 | 167 |

| | |
|---|---|
| Product length | 71 |

Primer pair 3

| | | | | | |
|---|---|---|---|---|---|
| Forward primer | GCCTGAAAGCCAGTAACCGT | 14 | 20 | 120 | 139 |
| Reverse primer | GGTGTTGCCAAAACGTTGCC | 15 | 20 | 189 | 170 |

| | |
|---|---|
| Product length | 70 |

Primer pair 4

| | | | | | |
|---|---|---|---|---|---|
| Forward primer | TATCACACTCGCCTGAAAGCC | 16 | 21 | 110 | 130 |
| Reverse primer | GCCAAAACGTTGCCACAAGAT | 17 | 21 | 183 | 163 |

| | |
|---|---|
| Product length | 74 |

Primer pair 5

| | | | | | |
|---|---|---|---|---|---|
| Forward primer | CCTGAAAGCCAGTAACCGTG | 18 | 20 | 121 | 140 |
| Reverse primer | TATGTTGCAGGTGTTGCCAAA | 19 | 21 | 198 | 178 |

| | |
|---|---|
| Product length | 78 |

Primer pair 6

| | | | | | |
|---|---|---|---|---|---|
| Forward primer | ACTCGCCTGAAAGCCAGTAAC | 20 | 21 | 116 | 136 |
| Reverse primer | GTGTTGCCAAAACGTTGCC | 21 | 19 | 188 | 170 |

| | |
|---|---|
| Product length | 73 |

Primer pair 7

| | | | | | |
|---|---|---|---|---|---|
| Forward primer | GGGAATGATATCACACTCGCCT | 6 | 22 | 102 | 123 |
| Reverse primer | TTGCCAAAACGTTGCCACAAG | 7 | 21 | 185 | 165 |

| | |
|---|---|
| Product length | 84 |

Primer pair 8

| | | | | | |
|---|---|---|---|---|---|
| Forward primer | ACCGAAAACACACACATAAACACT | 4 | 24 | 53 | 76 |
| Reverse primer | GGTTACTGGCTTTCAGGCGA | 5 | 20 | 137 | 118 |

| | |
|---|---|
| Product length | 85 |

Primer pair 9

| | | | | | |
|---|---|---|---|---|---|
| Forward primer | GTAACCGTGCGGGTTACAC | 22 | 19 | 132 | 150 |
| Reverse primer | ATTTATGTTGCAGGTGTTGCCA | 23 | 22 | 201 | 180 |

| | |
|---|---|
| Product length | 70 |

Primer pair 10

| | | | | | |
|---|---|---|---|---|---|
| Forward primer | GCCTGAAAGCCAGTAACCG | 24 | 19 | 120 | 138 |
| Reverse primer | CAGGTGTTGCCAAAACGTTGC | 25 | 21 | 191 | 171 |

| | |
|---|---|
| Product length | 72 |

20 primer sets were generated for Region B, as follows:
Primer pair 1

| | | | | | |
|---|---|---|---|---|---|
| Forward primer | CAGCGCTGTCTACCTCGTTA | 26 | 20 | 399 | 418 |
| Reverse primer | ACGGCTACTTCGCCTGATTT | 27 | 20 | 1203 | 1184 |

| | |
|---|---|
| Product length | 805 |

Primer pair 2

| | | | | | |
|---|---|---|---|---|---|
| Forward primer | GCGCTGTCTACCTCGTTAGT | 28 | 20 | 401 | 420 |
| Reverse primer | CACGCACTGACACAAGATTGG | 29 | 21 | 1396 | 1376 |

| | |
|---|---|
| Product length | 996 |

Primer pair 3

| | | | | | |
|---|---|---|---|---|---|
| Forward primer | TGAGTGCGTTCTTCATCACCA | 30 | 21 | 914 | 934 |
| Reverse primer | TACGGCTACTTCGCCTGATT | 31 | 20 | 1204 | 1185 |

| | |
|---|---|
| Product length | 291 |

Primer pair 4

| | | | | | |
|---|---|---|---|---|---|
| Forward primer | CAGCGCTGTCTACCTCGTT | 32 | 19 | 399 | 417 |
| Reverse primer | TGGTGATGAAGAACGCACTCA | 33 | 21 | 934 | 914 |

| | |
|---|---|
| Product length | 536 |

Primer pair 5

| | | | | | |
|---|---|---|---|---|---|
| Forward primer | TCAGGCGAAGTAGCCGTAAT | 34 | 20 | 1187 | 1206 |
| Reverse primer | ACGCACTGACACAAGATTGG | 35 | 20 | 1395 | 1376 |

| | |
|---|---|
| Product length | 209 |

Primer pair 6

| | | | | | |
|---|---|---|---|---|---|
| Forward primer | ATCAGGCGAAGTAGCCGTAA | 36 | 20 | 1186 | 1205 |
| Reverse primer | GGCACGCACTGACACAAGAT | 37 | 20 | 1398 | 1379 |

| | |
|---|---|
| Product length | 213 |

Primer pair 7

| | | | | | |
|---|---|---|---|---|---|
| Forward primer | TCAGCGCTGTCTACCTCGTT | 38 | 20 | 398 | 417 |
| Reverse primer | ATTACGGCTACTTCGCCTGA | 39 | 20 | 1206 | 1187 |

| | |
|---|---|
| Product length | 809 |

Primer pair 8

| | | | | | |
|---|---|---|---|---|---|
| Forward primer | AATCAGGCGAAGTAGCCGTA | 40 | 20 | 1185 | 1204 |
| Reverse primer | GGCACGCACTGACACAAGA | 41 | 19 | 1398 | 1380 |

| | |
|---|---|
| Product length | 214 |

Primer pair 9

| | | | | | |
|---|---|---|---|---|---|
| Forward primer | GCGCTGTCTACCTCGTTAGTA | 42 | 21 | 401 | 421 |
| Reverse primer | ACGGCTACTTCGCCTGATTTA | 43 | 21 | 1203 | 1183 |

| | |
|---|---|
| Product length | 803 |

Primer pair 10

| | | | | | |
|---|---|---|---|---|---|
| Forward primer | TCAGCGCTGTCTACCTCGT | 44 | 19 | 398 | 416 |
| Reverse primer | TACGGCTACTTCGCCTGATTT | 45 | 21 | 1204 | 1184 |

| | |
|---|---|
| Product length | 807 |

Primer pair 11

| | | | | | |
|---|---|---|---|---|---|
| Forward primer | GAGTGCGTTCTTCATCACCA | 46 | 20 | 915 | 934 |
| Reverse primer | TGGATGGACTCTTTTGAGGCA | 47 | 21 | 1068 | 1048 |

| | |
|---|---|
| Product length | 154 |

Primer pair 12

| | | | | | |
|---|---|---|---|---|---|
| Forward primer | TGAGTGCGTTCTTCATCACC | 48 | 20 | 914 | 933 |
| Reverse primer | CGGCTACTTCGCCTGATTTAC | 49 | 21 | 1202 | 1182 |

| | |
|---|---|
| Product length | 289 |

Primer pair 13

| | | | | | |
|---|---|---|---|---|---|
| Forward primer | AATCAGGCGAAGTAGCCGTAA | 50 | 21 | 1185 | 1205 |
| Reverse primer | GCACGCACTGACACAAGAT | 51 | 19 | 1397 | 1379 |

| | |
|---|---|
| Product length | 213 |

Primer pair 14

| | | | | | |
|---|---|---|---|---|---|
| Forward primer | TCAGCGCTGTCTACCTCGTTA | 52 | 21 | 398 | 418 |
| Reverse primer | GGTGATGAAGAACGCACTCA | 53 | 20 | 933 | 914 |

| | |
|---|---|
| Product length | 536 |

Primer pair 15

| | | | | | |
|---|---|---|---|---|---|
| Forward primer | AGCGCTGTCTACCTCGTTAGT | 54 | 21 | 400 | 420 |
| Reverse primer | TGGTGATGAAGAACGCACTC | 55 | 20 | 934 | 915 |

| | |
|---|---|
| Product length | 535 |

Primer pair 16

| | | | | | |
|---|---|---|---|---|---|
| Forward primer | GTCAGCGCTGTCTACCTCGT | 8 | 20 | 397 | 416 |
| Reverse primer | ACGGCTACTTCGCCTGATT | 9 | 19 | 1203 | 1185 |

| | |
|---|---|
| Product length | 807 |

Primer pair 17

| | | | | | |
|---|---|---|---|---|---|
| Forward primer | GTGCGTTCTTCATCACCATCA | 56 | 21 | 917 | 937 |
| Reverse primer | TCGCCTGATTTACCAGAAGGT | 57 | 21 | 1194 | 1174 |

| | |
|---|---|
| Product length | 278 |

Primer pair 18

| | | | | | |
|---|---|---|---|---|---|
| Forward primer | AAATCAGGCGAAGTAGCCGTA | 58 | 21 | 1184 | 1204 |
| Reverse primer | GGCACGCACTGACACAAG | 59 | 18 | 1398 | 1381 |

| | |
|---|---|
| Product length | 215 |

Primer pair 19

| | | | | | |
|---|---|---|---|---|---|
| Forward primer | TGGACTTTTTCGTCCCTTTCG | 60 | 21 | 577 | 597 |
| Reverse primer | TGATGGTGATGAAGAACGCAC | 61 | 21 | 937 | 917 |

| | |
|---|---|
| Product length | 361 |

Primer pair 20

| | | | | | |
|---|---|---|---|---|---|
| Forward primer | GGACTTTTTCGTCCCTTTCGT | 62 | 21 | 578 | 598 |
| Reverse primer | TTACGGCTACTTCGCCTGATTT | 63 | 22 | 1205 | 1184 |

| | |
|---|---|
| Product length | 628 |

DNA was extracted from Subdoligranulum isolates 7 and 1 as follows. Total bacterial genomic DNA extractions were performed using the QIAamp® Power Fecal DNA prep kit (Qiagen®, Germantown, MD) according to the manufacturer's instructions. Isolate 1 and 7 DNA were screened utilizing the primer sets and compared to a universal bacterial primer set, RpoB (global 16S bacterial primer; 5′->3′ rpoB1698 Forward: AACATCGGTTTGATCAAC (SEQ ID NO: 64) and rpoB2041 Reverse: CGTTGCATGTTGGTACCCAT (SEQ ID NO: 65))

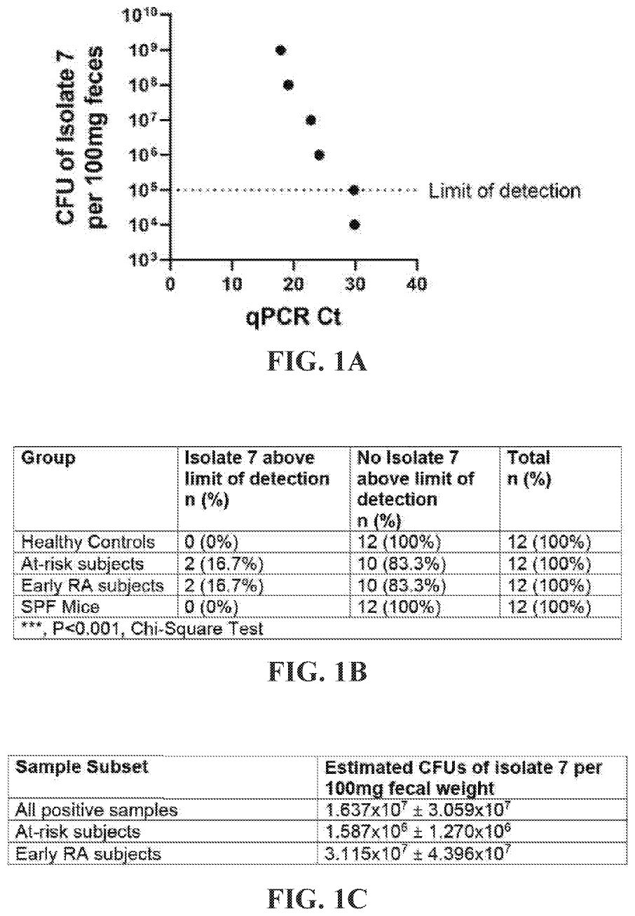

Surprisingly, of the 30 primer pairs designed and tested, only three primer sets demonstrated a strong fold change expression over RpoB and isolate 1. These three primer sets were flagged for further use and characterization of sensitivity to detect a low relative abundance of Subdoligranulum isolate 7 in a fecal sample. Next, known quantities of isolate 7 were spiked into a human fecal sample that had previously not been identified as containing Subdoligranulum to create a standard curve. The curve ranged from 1×10⁹ CFU of isolate 7 per 100 mg human feces to 0 CFU of isolate 7 per 100 mg of human feces. The three primer sets of interest were utilized against this standard curve and compared to RpoB as a control. The best primer set was identified (primer pair 8) and the limit of detection of 1×10⁵ CFU of isolate 7 per 100 mg feces was established based on qPCR Ct versus CFUs of Isolate 7 (FIG. 1A). A line of best fit was established for the curve (R²=0.9982) and utilized for regression analysis of human and murine samples.

The qPCR assay demonstrates specificity towards isolate 7 and sensitivity down to 10,000 bacteria per 10 mg of fecal bacteria (FIG. 1A).

### Example 2: Subdoligranulum Isolate 7 is Detectable in the Feces of Individuals in the at-Risk Period and Early Stages of RA

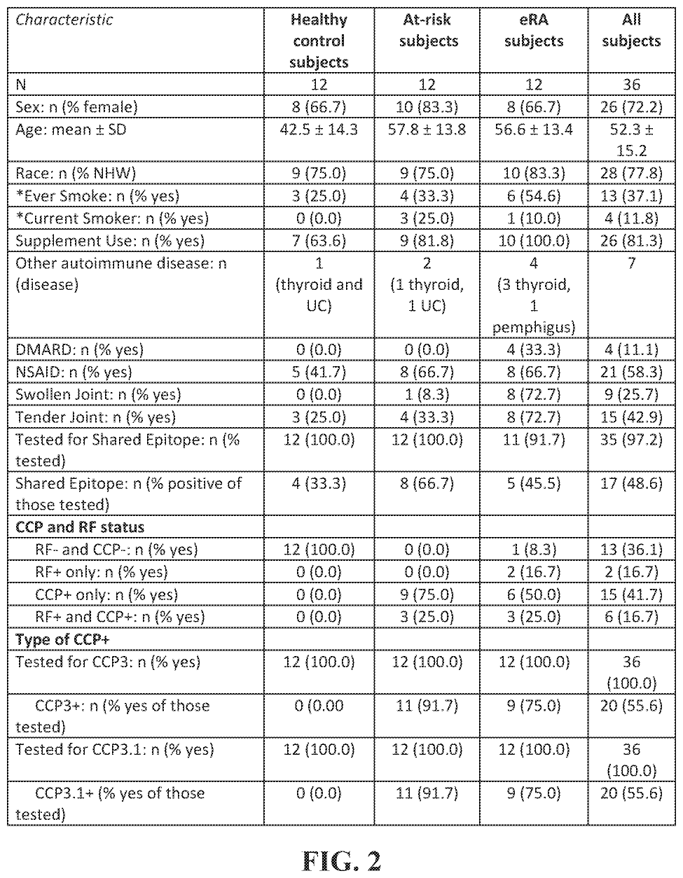

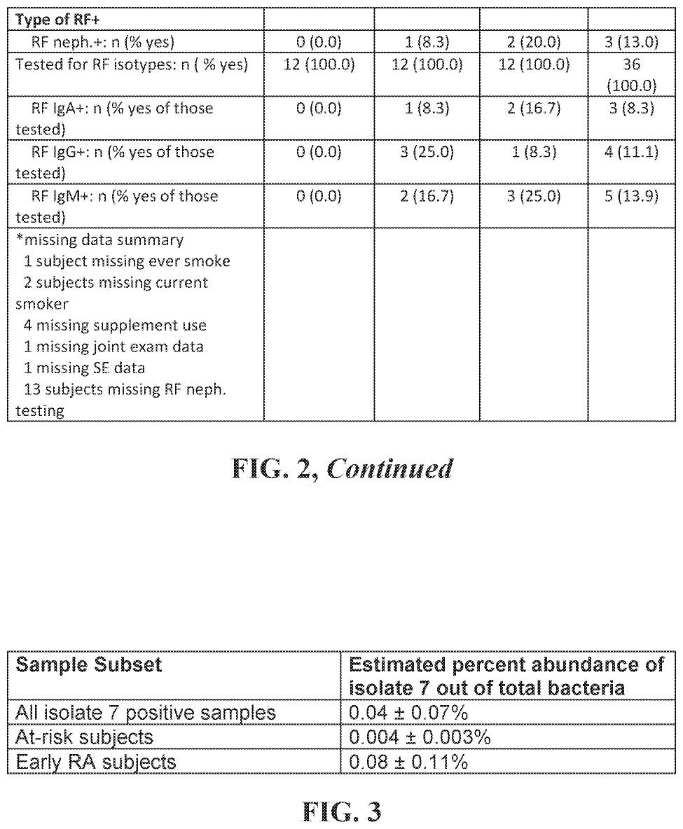

The feces of healthy human control subjects (n=12), individuals at-risk for RA (n=12), and individuals with early RA (<1 year from diagnosis, n=12; FIG. 2) were screened for the presence of isolate 7 in 100 mg of feces. These samples were compared to the standard curve and through regression analysis, the CFUs of isolate 7 per 100 mg of fecal weight was established.

The prevalence of isolate 7 above the limit of detection was 16.7% in the at-risk and early RA groups, but 0% in healthy controls (P<0.001, FIG. 1B). The abundance of isolate 7 varied among the positive samples, with an average of 1.637×10⁷±3.059×10⁷ CFUs per positive sample (FIG. 1C).

Previous studies have estimated that 100 mg of fecal weight contains roughly 4.1×10¹⁰ bacteria (Stephen, 1980, J Med Microbiol 13:45-56), indicating that the positive samples from this study have an average of 0.04±0.07% abundance of isolate 7 in their microbiomes (FIG. 3).

Twelve (12) mice from our specific pathogen free colony were similarly tested for the presence of isolate 7. Isolate 7 was undetectable in the feces of all the mice (FIG. 1B). Thus, the presence of isolate 7 appears to be restricted to a subgroup of individuals at-risk for, and with, RA.

### Example 3: Prevalence and Comparison of the Fecal Concentration of S. dido7 Among Healthy Controls Vs. Individuals at-Risk and with Early RA

In this study, we sought to assess the prevalence and compare the fecal concentration of S. dido7 among healthy controls vs. individuals at-risk and with early RA and its association with CCP3 antibodies and biomarkers of intestinal permeability, zonulin and soluble CD14.

Stool and sera were prospectively collected from 3 groups: 1) ‘At-Risk’ human individuals, defined as those with positive CCP3 with no history of, nor current, IA (n=54), 2) individuals with CCP3(+) RA based on 2010 ACR/EULAR criteria within 12 months of diagnosis (n=5) and 3) controls without IA or RF/CCP3 positivity (n=32). Exclusion criteria included antibiotics in the 3 months prior to sample collection. Fecal DNA was extracted using a commercial kit (Qiagen). Quantitative PCR was used to create a standard curve using known concentrations of S. dido7 to allow for calculation of strain-specific fecal levels in individual samples. Serum CCP3 ELISA (Werfen) was used to measure CCP IgG. Serum ELISAs (Cusabio) were used to quantitate serum zonulin and soluble CD14.

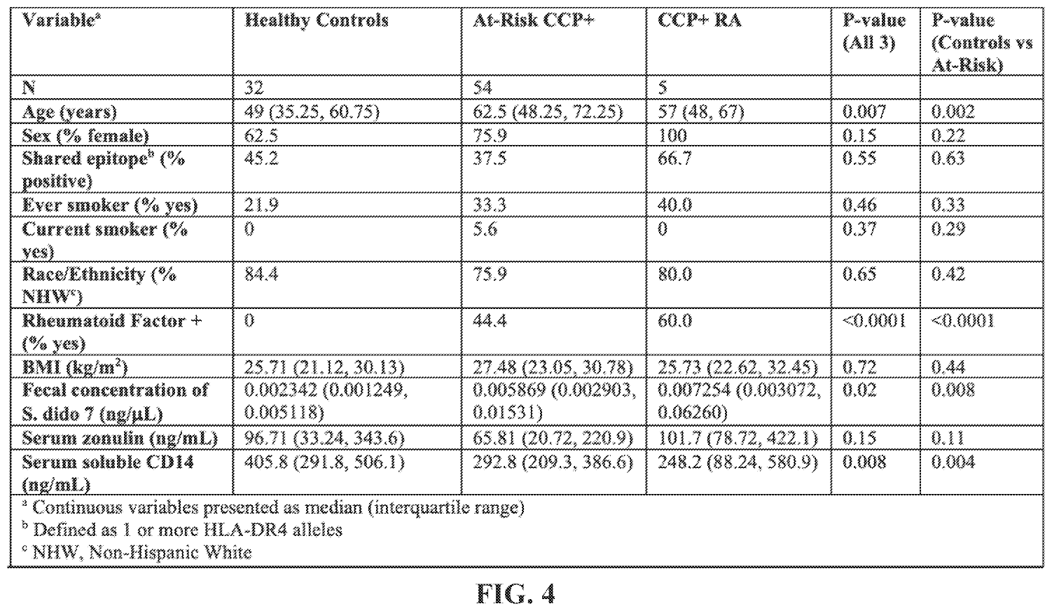

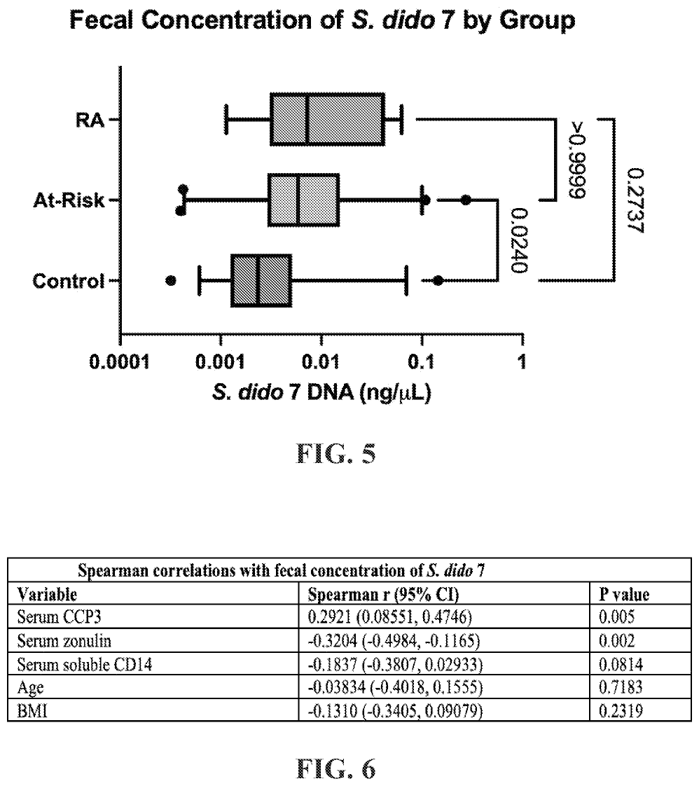

Demographics of the three groups of human test subjects are summarized in FIG. 4. There were no significant differences across the groups apart from age. Fecal concentration of S. dido7 was 2.5-fold higher in At-Risk individuals and 3.1-fold higher in early RA individuals compared to controls (p-0.02; FIG. 5). Higher serum CCP3 levels significantly correlated with higher fecal S. dido 7 concentration (p=0.005; FIG. 6). Serum CD14 but not zonulin was significantly lower in At-Risk individuals and early RA individuals (p=0.008; FIG. 4). Higher zonulin significantly correlated with lower fecal S. dido7 concentration (p=0.002; FIG. 6). There was no significant correlation between fecal S. dido7 and serum soluble CD14 or age (FIG. 6).

Although embodiments have been described in detail with reference to examples above, it is understood that various modifications can be made without departing from the spirit of this disclosure, and would readily be known to the skilled artisan.

These data, along with the prior data demonstrating arthritogenic potential of this bacterial strain, suggest S. dido7 may play an important mechanistic role in a subset of individuals during development of CCP autoantibodies and subsequent RA.

These and other aspects disclosed herein, including the exemplary diagnostic methods, treatment methods, and uses, will be apparent from the teachings contained herein.

1. A method for diagnosing rheumatoid arthritis (RA) in a subject, the method comprising:
a) obtaining a biological sample from the subject;
b) detecting presence or absence of Subdoligranulum didolesgii strain D8 in the biological sample, wherein the bacteria comprises SEQ ID NO: 2 (Region A) and/or SEQ ID NO: 3 (Region B), and
c) diagnosing the subject with RA when Subdoligranulum didolesgii strain D8 bacteria is detected in the biological sample.

2. The method of claim 1, wherein the presence of Subdoligranulum didolesgii strain D8 is detected by polymerase chain reaction (PCR) analysis or sequencing.

3. The method of claim 1 or claim 2, wherein the presence of Subdoligranulum didolesgii strain D8 is detected by quantitative PCR (qPCR) analysis.

4. The method of claim 2 or 3, wherein the detecting step is specific for detection of a sequence within SEQ ID NO: 2 (Region A).

5. The method of claim 4, wherein detecting a sequence within SEQ ID NO: 2 (Region A) uses an oligonucleotide primer pair of Primer Pair 8:

| | forward primer |
|---|---|
| (SEQ ID NO: 4) | |
| | ACCGAAAACACACACATAAACACT |
| | and |
| | reverse primer |
| (SEQ ID NO: 5) | |
| | GGTTACTGGCTTTCAGGCGA |
| | or |
| | Primer Pair 7: |
| | forward primer |
| (SEQ ID NO: 6) | |
| | GGGAATGATATCACACTCGCCT |
| | and |
| | reverse primer |
| (SEQ ID NO: 7) | |
| | TTGCCAAAACGTTGCCACAAG. |

6. The method of claim 4, wherein detecting a sequence within SEQ ID NO: 2 (Region A) uses an oligonucleotide primer pair of Primer Pair 8:

| | forward primer |
|---|---|
| (SEQ ID NO: 4) | |
| | ACCGAAAACACACACATAAACACT |
| | and |
| | reverse primer |
| (SEQ ID NO: 5) | |
| | GGTTACTGGCTTTCAGGCGA. |

7. The method of any one of claims 2-6, wherein the detecting step comprises detection of a sequence within SEQ ID NO: 3 (Region B) or further comprises detection of a sequence within SEQ ID NO: 3 (Region B).

8. The method of claim 7, wherein detecting a sequence within SEQ ID NO: 3 (Region B) uses an oligonucleotide primer pair of Primer Pair 16:

| | forward primer |
|---|---|
| (SEQ ID NO: 8) | |
| | GTCAGCGCTGTCTACCTCGT |
| | and |
| | reverse primer |
| (SEQ ID NO: 9) | |
| | ACGGCTACTTCGCCTGATT. |

9. The method of claim 2 or 3, wherein the detecting step comprises detection of at least one sequence within SEQ ID NO: 2 (Region A) and detection of at least one sequence within SEQ ID NO: 3 (Region B).

10. The method of claim 9, wherein detection of at least one sequence within SEQ ID NO: 2 (Region A) uses Primer Pair 8:

| | forward primer |
|---|---|
| (SEQ ID NO: 4) | |
| | ACCGAAAACACACACATAAACACT |
| | and |
| | reverse primer |
| (SEQ ID NO: 5) | |
| | GGTTACTGGCTTTCAGGCGA |
| | and |
| | Primer Pair 7: |
| | forward primer |
| (SEQ ID NO: 6) | |
| | GGGAATGATATCACACTCGCCT |
| | and |
| | reverse primer |
| (SEQ ID NO: 7) | |
| | TTGCCAAAACGTTGCCACAAG |

and detection of at least one sequence within SEQ ID NO: 3 (Region B) uses Primer Pair 16:

| | forward primer |
|---|---|
| (SEQ ID NO: 8) | |
| | GTCAGCGCTGTCTACCTCGT |
| | and |
| | reverse primer |
| (SEQ ID NO: 9) | |
| | ACGGCTACTTCGCCTGATT. |

11. The method of any one of the preceding claims, wherein the rheumatoid arthritis is early rheumatoid arthritis.

12. The method of any one of the preceding claims, wherein the biological sample is a fecal sample, a rectal swab, serum, plasma, peripheral blood cells, or an intestinal tissue biopsy.

13. The method of any one of the preceding claims, wherein detecting the presence of Subdoligranulum didolesgii strain D8 is assessed relative to a control sample from a subject not diagnosed with RA.

14. The method of any one of the preceding claims, further comprising administering an RA treatment to the subject diagnosed with RA.

15. The method of claim 14, wherein the RA treatment comprises administering to the subject one or more compounds selected from: a non-steroidal inflammatory drug; a corticosteroid; a disease-modifying antirheumatic drug; and a biologic response modifier.

16. The method of 15, wherein:
a. the non-steroidal inflammatory drug is selected from: ibuprofen; naproxen sodium; celecoxib; diclofenac; fenoprofen; flurbiprofen; indomethacin;
ketorolac; mefenamic acid; meloxicam; oxaprozin; piroxicam; and sulindac;
b. the corticosteroid is selected from: prednisone; bethamethasone; and prednisolone; triamcinolone; methylprednisolone; and dexamethasone;
c. the disease-modifying antirheumatic drug is selected from: methotrexate; leflunomide; hydroxychloroquine; and sulfasalazine; and
d. the biologic response modifier is selected from: abatacept; adalimumab; anakinra; baricitinib; certolizumab; etanercept; golimumab; infliximab; rituximab; sarilumab; tocilizumab; and tofacitinib.

17. A method for diagnosing risk of rheumatoid arthritis (RA) in a subject, the method comprising:
a) obtaining a biological sample from the subject;
b) detecting presence or absence of Subdoligranulum didolesgii strain D8 in the biological sample, wherein the bacteria comprises SEQ ID NO: 2 (Region A) and/or SEQ ID NO: 3 (Region B), and
c) diagnosing the subject as at risk of developing RA when Subdoligranulum didolesgii strain D8 bacteria is detected in the biological sample.

18. The method of claim 17, wherein the presence of Subdoligranulum didolesgii strain D8 is detected by polymerase chain reaction (PCR) analysis or sequencing.

19. The method of claim 17 or claim B2, wherein the presence of Subdoligranulum didolesgii strain D8 is detected by quantitative PCR (qPCR) analysis.

20. The method of claim 18 or 19, wherein the detecting step is specific for detection of a sequence within SEQ ID NO: 2 (Region A).

21. The method of claim 20, wherein detecting a sequence within SEQ ID NO: 2 (Region A) uses an oligonucleotide primer pair of Primer Pair 8:

| | forward primer |
|---|---|
| (SEQ ID NO: 4) | |
| | ACCGAAAACACACACATAAACACT |
| | and |
| | reverse primer |
| (SEQ ID NO: 5) | |
| | GGTTACTGGCTTTCAGGCGA |
| | or |
| | Primer Pair 7: |
| | forward primer |
| (SEQ ID NO: 6) | |
| | GGGAATGATATCACACTCGCCT |
| | and |
| | reverse primer |
| (SEQ ID NO: 7) | |
| | TTGCCAAAACGTTGCCACAAG. |

22. The method of claim 20, wherein detecting a sequence within SEQ ID NO: 2 (Region A) uses an oligonucleotide primer pair of Primer Pair 8:

| | forward primer |
|---|---|
| (SEQ ID NO: 4) | |
| | ACCGAAAACACACACATAAACACT |
| | and |
| | reverse primer |
| (SEQ ID NO: 5) | |
| | GGTTACTGGCTTTCAGGCGA. |

23. The method of any one of claims 18-22, wherein the detecting step comprises detection of a sequence within SEQ ID NO: 3 (Region B) or further comprises detection of a sequence within SEQ ID NO:3 (Region B).

24. The method of claim 23, wherein detecting a sequence within SEQ ID NO: 3 (Region B) uses an oligonucleotide primer pair of Primer Pair 16:

| | forward primer |
|---|---|
| (SEQ ID NO: 8) | |
| | GTCAGCGCTGTCTACCTCGT |
| | and |
| | reverse primer |
| (SEQ ID NO: 9) | |
| | ACGGCTACTTCGCCTGATT. |

25. The method of claim 18 or 19, wherein the detecting step comprises detection of at least one sequence within SEQ ID NO: 2 (Region A) and detection of at least one sequence within SEQ ID NO: 3 (Region B).

26. The method of claim 25, wherein detection of at least one sequence within SEQ ID NO: 2 (Region A) uses Primer Pair 8:

| | forward primer |
|---|---|
| (SEQ ID NO: 4) | |
| | ACCGAAAACACACACATAAACACT |
| | and |
| | reverse primer |
| (SEQ ID NO: 5) | |
| | GGTTACTGGCTTTCAGGCGA |
| | and |
| | Primer Pair 7: |
| | forward primer |
| (SEQ ID NO: 6) | |
| | GGGAATGATATCACACTCGCCT |
| | and |
| | reverse primer |
| (SEQ ID NO: 7) | |
| | TTGCCAAAACGTTGCCACAAG |

and detection of at least one sequence within SEQ ID NO: 3 (Region B) uses Primer Pair 16:

| | forward primer |
|---|---|
| (SEQ ID NO: 8) | |
| | GTCAGCGCTGTCTACCTCGT |
| | and |
| | reverse primer |
| (SEQ ID NO: 9) | |
| | ACGGCTACTTCGCCTGATT. |

27. The method of any one of claims 17-26, wherein the biological sample is a fecal sample, a rectal swab, serum, plasma, peripheral blood cells, or an intestinal tissue biopsy.

28. The method of any one of claim 17-27, wherein detecting the presence of Subdoligranulum didolesgii strain D8 is assessed relative to a control sample from a subject not diagnosed with RA and not at risk of RA.

29. The method of any one of claims 17-28, further comprising administering a prophylactic RA treatment to the subject diagnosed as at risk of developing RA.

30. A method of detecting Subdoligranulum didolesgii strain D8 in a subject, the method comprising:
a) obtaining a fecal sample from the subject;
b) detecting presence or absence of Subdoligranulum didolesgii strain D8 in the fecal sample, wherein the bacteria comprises SEQ ID NO: 2 (Region A) and/or SEQ ID NO: 3 (Region B).

31. The method of claim 30, wherein the presence of Subdoligranulum didolesgii strain D8 is detected by polymerase chain reaction (PCR) analysis or nucleic acid sequencing.

32. The method of claim 30 or claim B2, wherein the presence of Subdoligranulum didolesgii strain D8 is detected by quantitative PCR (qPCR) analysis.

33. The method of claim 31 or 32, wherein the detecting step is specific for detection of a sequence within SEQ ID NO: 2 (Region A).

34. The method of claim 33, wherein detecting a sequence within SEQ ID NO: 2 (Region A) uses an oligonucleotide primer pair of Primer Pair 8:

| | forward primer |
|---|---|
| (SEQ ID NO: 4) | |
| | ACCGAAAACACACACATAAACACT |
| | and |
| | reverse primer |
| (SEQ ID NO: 5) | |
| | GGTTACTGGCTTTCAGGCGA |
| | or |
| | Primer Pair 7: |
| | forward primer |
| (SEQ ID NO: 6) | |
| | GGGAATGATATCACACTCGCCT |
| | and |
| | reverse primer |
| (SEQ ID NO: 7) | |
| | TTGCCAAAACGTTGCCACAAG. |

35. The method of claim 33, wherein detecting a sequence within SEQ ID NO: 2 (Region A) uses an oligonucleotide primer pair of Primer Pair 8:

| | forward primer |
|---|---|
| (SEQ ID NO: 4) | |
| | ACCGAAAACACACACATAAACACT |
| | and |
| | reverse primer |
| (SEQ ID NO: 5) | |
| | GGTTACTGGCTTTCAGGCGA. |

36. The method of any one of claims 31-33, wherein the detecting step comprises detection of a sequence within SEQ ID NO: 3 (Region B) or further comprises detection of a sequence within SEQ ID NO:3 (Region B).

37. The method of claim 36, wherein detecting a sequence within SEQ ID NO: 3 (Region B) uses an oligonucleotide primer pair of Primer Pair 16:

| | forward primer |
|---|---|
| (SEQ ID NO: 8) | |
| | GTCAGCGCTGTCTACCTCGT |
| | and |
| | reverse primer |
| (SEQ ID NO: 9) | |
| | ACGGCTACTTCGCCTGATT. |

38. The method of claim 31 or 32, wherein the detecting step comprises detection of at least one sequence within SEQ ID NO: 2 (Region A) and detection of at least one sequence within SEQ ID NO: 3 (Region B).

39. The method of claim 38, wherein detection of at least one sequence within SEQ ID NO: 2 (Region A) uses Primer Pair 8:

| | forward primer |
|---|---|
| (SEQ ID NO: 4) | |
| | ACCGAAAACACACACATAAACACT |
| | and |
| | reverse primer |
| (SEQ ID NO: 5) | |
| | GGTTACTGGCTTTCAGGCGA |
| | and |
| | Primer Pair 7: |
| | forward primer |
| (SEQ ID NO: 6) | |
| | GGGAATGATATCACACTCGCCT |
| | and |
| | reverse primer |
| (SEQ ID NO: 7) | |
| | TTGCCAAAACGTTGCCACAAG |

and detection of at least one sequence within SEQ ID NO: 3 (Region B) uses Primer Pair 16:

| | forward primer |
|---|---|
| (SEQ ID NO: 8) | |
| | GTCAGCGCTGTCTACCTCGT |
| | and |
| | reverse primer |
| (SEQ ID NO: 9) | |
| | ACGGCTACTTCGCCTGATT. |

40. A kit for the detection of Subdoligranulum didolesgii strain D8 in a biological sample from a subject, the kit comprising at least one PCR primer selected from the group consisting of ACCGAAAACACACACATAAACACT (SEQ ID NO: 4), GGTTACTGGCTTTCAGGCGA (SEQ ID NO: 5), GGGAATGATATCACACTCGCCT (SEQ ID NO: 6), TTGCCAAAACGTTGCCACAAG (SEQ ID NO: 7), GTCAGCGCTGTCTACCTCGT (SEQ ID NO: 8), and ACGGCTACTTCGCCTGATT (SEQ ID NO: 9).
